(19) United States
(12) Patent Application Publication
(10) Pub. No.: US 2026/0078101 A1
(43) Pub. Date: Mar. 19, 2026

(54) RECOVERY OF MALEIC ANHYDRIDE

(71) Applicant: HUNTSMAN PETROCHEMICAL LLC, The Woodlands, TX (US)

(72) Inventor: Michael F UEHLINGER, The Woodlands, TX (US)

(21) Appl. No.: 19/108,526

(22) PCT Filed: Aug. 23, 2023
(86) PCT No.: PCT/US2023/030884

§ 371 Date: Mar. 4, 2025

Related U.S. Application Data
(60) Provisional application No. 63/405,166, filed on Sep. 9, 2022.

(51) Int. Cl. C07D 307/60

(52) U.S. Cl. CPC C07D 307/60

(57) ABSTRACT

A process and system for extracting maleic anhydride from a gaseous mixture where the gaseous mixture is contacted with an extraction solvent capable of absorbing maleic anhydride at temperatures between about −60° C. and 250° C. and at pressures up to about 50 psi above atmospheric pressure.

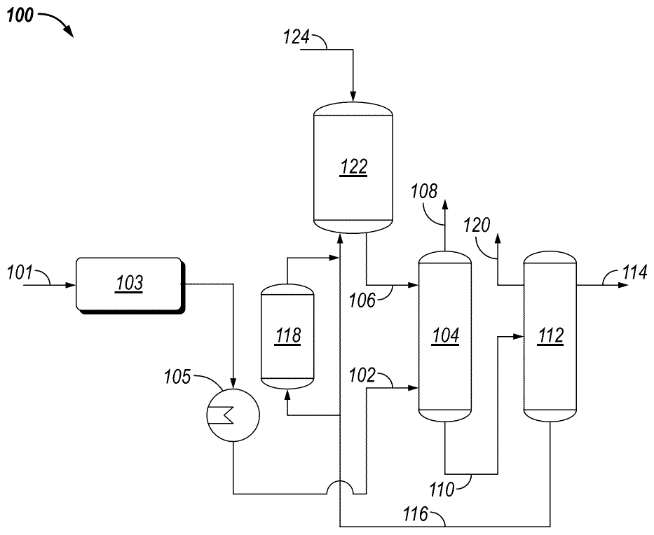

### CROSS REFERENCE TO RELATED APPLICATIONS

This application claims priority to U.S. Provisional Application 63/405,166 filed Sep. 9, 2022. The noted application(s) are incorporated herein by reference.

### STATEMENT REGARDING FEDERALLY SPONSORED RESEARCH OR DEVELOPMENT

Not applicable.

### FIELD

The present disclosure generally relates to the recovery of maleic anhydride from a gaseous mixture containing the maleic anhydride, particularly by use of an extraction solvent comprising a phosphate ester.

### BACKGROUND

Maleic anhydride (cis-butanedioic anhydride) is used alone or in combination with other acids in the manufacture of alkyd and polyester resins. It is also a versatile intermediate for chemical synthesis. Global demand for maleic anhydride has been increasing at a steady rate and it is anticipated this will continue over the next several years.

Maleic anhydride can be prepared commercially by contacting a feed gas comprising molecular oxygen and a suitable hydrocarbon including, but not limited to, n-butane and butene, with a vanadium-phosphorus-oxygen fixed-bed or fluidized catalyst in a reactor. The reaction product gas that is discharged from the reactor contains maleic anhydride together with oxidation by-products such as carbon monoxide, carbon dioxide, water vapor, acrylic and acetic acids, along with inert gases present in air when air is used as the source of molecular oxygen.

Various methods of recovering maleic anhydride from the reaction product gas exist in the field. In at least one example, the maleic anhydride is recovered by direct condensation of the reaction product gas or by scrubbing the gas with water and dehydrating the resulting aqueous mixture by azeotropic distillation with xylene. However, due to increased product yields, the preferred method of recovery includes selectively absorbing the maleic anhydride in a suitable solvent and subsequently stripping the maleic anhydride from the enriched solvent to obtain a crude maleic anhydride product.

Currently, the solvent typically used to selectively absorb maleic anhydride is dibutyl phthalate (CAS #84-74-2) (DBP). However, DBP is undergoing various reviews by regional governments due to toxicological concerns which could prevent its use in future isolation and recovery methods. Accordingly, there is a continuous need for the development of improved solvents which have minimal toxicity and are capable of replacing DBP in selectively absorbing maleic anhydride from reaction product gases.

### SUMMARY

The present disclosure describes a process for recovering maleic anhydride from a gaseous mixture containing the maleic anhydride. The process includes a step (a) of contacting the gaseous mixture with an extraction solvent comprising a phosphate ester wherein the contacting comprises transferring at least a portion of the maleic anhydride from the gaseous mixture to the extraction solvent. The process may further include a step (b) of subjecting the solvent effluent stream obtained in step (a) to stripping to produce a stream comprising a crude maleic anhydride product and a regenerable extraction solvent substantially free of maleic anhydride and comprising the extraction solvent. The process may also include a step (c) of recycling at least a portion of the regenerable extraction solvent comprising the extraction solvent to step (a).

The present disclosure also provides a system for the recovery of maleic anhydride from a gaseous mixture containing the maleic anhydride. The system includes an absorption zone operable to contact the gaseous mixture with an extraction solvent comprising a phosphate ester to transfer the maleic anhydride from the gaseous mixture to the extraction solvent, and a stripping zone operable to separate the maleic anhydride from the extraction solvent, where the absorption zone includes a solvent storage tank comprising the extraction solvent.

### BRIEF DESCRIPTION OF THE DRAWINGS

The FIGURE depicts a process flow diagram summarizing a process and system for recovering maleic anhydride according to certain embodiments described herein.

### DETAILED DESCRIPTION

The present disclosure generally provides a process for the extraction of maleic anhydride from a gaseous mixture by contacting the gaseous mixture with an extraction solvent comprising a phosphate ester to produce a gas stream substantially free of maleic anhydride and a solvent effluent stream comprising absorbed maleic anhydride and the extraction solvent. The absorbed maleic anhydride may be recovered from the solvent effluent stream in a subsequent separation step to produce a crude maleic anhydride product. In certain embodiments, the extraction solvent is regenerable in that the maleic anhydride is released from the solvent effluent stream, and the extraction solvent can be recycled to contact additional gaseous mixtures. It has been surprisingly found that because the extraction solvent comprising the phosphate ester can effectively absorb maleic anhydride from the gaseous mixture, it may be used in place of current state of the art liquid extraction solvents comprising dibutyl phthalate alleviating toxicity concerns but without a loss in performance. In addition, in current state of the art processes, the gaseous mixture leaving the reactor after the partial oxidation of C₄-hydrocarbons must typically be cooled in one or more heat exchangers prior to recovery. However, this step is known to cause fouling in the heat exchangers due to various contaminants contained within the gaseous mixture thus requiring additional cleaning steps and increased operation costs. The extraction solvent of the present disclosure is capable of recovering the maleic anhydride from gaseous mixtures at higher temperatures thereby eliminating this cooling step resulting in a more efficient and cost-effective process. Finally, because of the relatively high-volatility of the extraction solvent of the present disclosure, maleic anhydride absorbed therein may be separated from the extraction solvent using less energy.

Before explaining aspects of the present disclosure in detail, it is to be understood that the present disclosure is not limited in its application to the details of construction and the arrangement of components or steps, or methodologies set forth in the following description. The present disclosure is capable of other embodiments or of being practiced or carried out in various ways. Also, it is to be understood that the phraseology and terminology employed herein is for the purpose of description and should not be regarded as limiting.

If appearing herein, the term “comprising” and derivatives thereof are not intended to exclude the presence of any additional component, step, or procedure, whether or not the same is disclosed herein. In order to avoid any doubt, all compositions claimed herein through use of the term “comprising” may include any additional additive, adjuvant, or compound, unless stated to the contrary. In contrast, the term, “consisting essentially of” if appearing herein, excludes from the scope of any succeeding recitation any other component, step, or procedure, except those that are not essential to operability and the term “consisting of”, if used, excludes any component, step or procedure not specifically delineated or listed. The terms “or” and “and/or”, unless stated otherwise, refer to the listed members individually as well as in any combination. For example, the expressions A or B and A and/or B refer to A alone, B alone, or to both A and B.

The articles “a” and “an” are used herein to refer to one or to more than one (i.e., to at least one) of the grammatical objects of the article. By way of example, “a solvent” means one solvent or more than one solvent. The phrases “in one embodiment”, “according to one embodiment” and the like generally mean the feature, structure, or characteristic following the phrase is included in at least one embodiment of the present disclosure and may be included in more than one embodiment of the present disclosure. Importantly, such phrases do not necessarily refer to the same embodiment. If the specification states a component or feature “may”, “can”, “could”, or “might” be included or have a characteristic, that component or feature is not required to be included or have the characteristic.

The terms “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the present disclosure.

The term “about” as used herein can allow for a degree of variability in a value or range, for example, it may be within 10%, within 5%, or within 1% of a stated value or of a stated limit of a range.

The use of ordinal number terminology (i.e., “first”, “second”, “third”, “fourth”, etc.) is solely for the purpose of differentiating between two or more items and, unless otherwise stated, is not meant to imply any sequence or order or importance to one item over another or any order of addition.

Values expressed in a range format should be interpreted in a flexible manner to include not only the numerical values explicitly recited as the limits of the range, but to also include all the individual numerical values or sub-ranges encompassed within that range as if each numerical value and sub-range is explicitly recited. For example, a range such as from 1 to 6, should be considered to have specifically disclosed sub-ranges, such as, from 1 to 3, from 2 to 4, from 3 to 6, etc., as well as individual numbers within that range, for example, 1, 2, 3, 4, 5, and 6. This applies regardless of the breadth of the range.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “optional” or “optionally” means that the subsequently described event or circumstance may or may not occur, and that the description includes instances where said event or circumstance occurs and instances where it does not.

The term “aryl” means an unsaturated aromatic hydrocarbon ring system radical. Aryl ring systems include phenyl, naphthalenyl, azulenyl, anthracenyl and the like.

The term “regenerable” means a medium may be subjected to a treatment to recover its functions and qualities close to those of origin.

The term “absorption” or variants thereof generally means a reversible process whereby some components of a gas mixture are surrounded by and interact with solvent molecules to undergo solvation and form a solution.

The term “stripping” is taken to include general methods for removing, separating or (forcefully) expelling gaseous compounds from a liquid stream.

The term “substantially free” refers to a composition in which a particular constituent or moiety is present in an amount that has no material effect on the overall composition. In some embodiments, “substantially free” may refer to a composition in which the particular constituent or moiety is present in the composition in an amount of less than about 5 wt. %, or less than about 4 wt. %, or less than about 3 wt. % or less than about 2 wt. % or less than about 1 wt. %, or less than about 0.5 wt. %, or less than about 0.1 wt. %, or less than about 0.05 wt. %, or even less than about 0.01 wt. % based on the total weight of the composition, or that no amount of that particular constituent or moiety is present in the respective composition.

According to one embodiment, the present disclosure provides a process for recovering maleic anhydride from a gaseous mixture containing the maleic anhydride, the process including a step (a) of contacting the gaseous mixture with an extraction solvent comprising a phosphate ester where the contacting comprises transferring at least a portion of the maleic anhydride from the gaseous mixture to the extraction solvent. In some embodiments, the contacting step (a) may be conducted in an absorber to provide an exhaust gas stream substantially free of maleic anhydride and a solvent effluent stream comprising the extraction solvent, and maleic anhydride.

A representative gaseous mixture includes a reaction product gas obtained from the oxidation of a gas-phase hydrocarbon, such as n-butane or butene, over a fixed-bed or fluidized bed of catalyst, such as a vanadium-phosphorus-oxygen catalyst, in a reactor. The reaction product gas may include from about 0.5-2.5 mole % of maleic anhydride, about 4-20 mole % water vapor along with nitrogen, oxygen, unreacted hydrocarbons and oxidation by-products
including those described above (for e.g., carbon monoxide, carbon dioxide, acrylic, maleic and acetic acids). The maleic anhydride and a portion of the oxidation-by products are absorbed in the extraction solvent (along with other various contaminants such as carboxylic acids and organic and mineral phosphates) while the remaining oxidation-by products and inert gases are contained within the exhaust gas stream.

In one embodiment, the phosphate ester is a compound having the formula

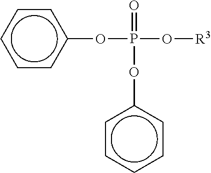

where R³ is a linear or branched alkyl group having up to 22 carbon atoms, an aryl group, or an -aryl-(C₁-C₁₀)alkyl group.

In one embodiment, the phosphate ester is a mono alkyl diphenyl phosphate ester where R³ is a linear or branched alkyl group containing up to 22 carbon atoms, more specifically 6 to 14 carbon atoms. In one embodiment, R³ is a branched alkyl group containing 6 to 10 carbon atoms.

Examples of mono alkyl diphenyl phosphate esters which may be used include without limitation, 2-ethylhexyl diphenyl phosphate, nonyl diphenyl phosphate, iso-octyl diphenyl phosphate, 2-methylpentyl diphenyl phosphate, 2-n-propylheptyl diphenyl phosphate, 2-butyloctyl diphenyl phosphate, 2-ethylbutyl diphenyl phosphate, isodecyl diphenyl phosphate and mixtures thereof.

In another embodiment, the phosphate ester is a compound where R³ is an -aryl-(C₁-C₁₀)alkyl group, such as an -aryl-(C₁-C₄)alkyl group. In one embodiment, R³ is tolyl, cresyl, xylyl, t-butylphenyl or isopropylphenyl.

In another embodiment, the extraction solvent may further comprise an organic solvent. The organic solvent may include, but is not limited to, maleates, phthalates, carbonates, benzoates, ketones, aromatics, anhydrides, halogenated hydrocarbons, halogenated oxy hydrocarbons, ether acetates, naphthalenes, ethers, and non-phosphate esters. More specifically, the organic solvent may include dimethyl maleates, diethyl maleates, propylene carbonate, propyl benzoate, isobutyl benzoate, isophorone, e-caprolactone, isobutyl heptyl ketone, di-normal amyl ketone, di-isoamyl ketone, hexyl benzene, mixed aromatics, n-valeric anhydride, C₉ alkyl acetate ester, C₁₀ alky acetate ester, 1,4-butanediol diacetate, malonic acid-dipropyl ester, dimethyl phthalates, and esters of C₅ to C₁₀ neo acids and mono-polyhydric alcohols. In one embodiment, the extraction solvent is substantially free of such organic solvents.

In some embodiments, the contacting step (a) may be conducted at a temperature greater than about −60° C., or greater than about −30° C., or greater than about 10° C. or greater than about 50° C. or greater than about 100° C., or greater than about 120° C., or greater than about 140° C., or greater than about 160° C., or greater than about 180° C., or greater than about 200° C. or greater than about 220° C. In other embodiments, the contacting step (a) may be conducted at a temperature less than about 250° C., or less than about 240° C., or less than about 230° C., or less than about 220° C., or less than about 210° C., or less than about 200° C., or less than about 190° C., or less than about 180° C. or less than about 170° C. In still other embodiments, the contacting step (a) may be conducted at a temperature within a range from about −60° C. to about 250° C.

In some embodiments, the contacting step (a) may be conducted at a pressure greater than atmospheric pressure, such as a pressure up to about 10 psi above atmospheric pressure, or up to about 20 psi above atmospheric pressure, or up to about 30 psi above atmospheric pressure, or up to about 40 psi above atmospheric pressure, or up to about 50 psi above atmospheric pressure.

In another embodiment, the process further includes a step (b) of subjecting the solvent effluent stream obtained in step (a) to stripping to produce a first stream comprising a crude maleic anhydride product and a regenerable extraction solvent substantially free of maleic anhydride and comprising the extraction solvent and contaminants. In some embodiments, step (b) may be conducted in a distillation column. Therefore, in one embodiment, the extraction solvent is capable of surviving distillation at temperatures above 180° C. to approximately 250° C. in order to sufficiently separate the maleic anhydride from the solvent effluent stream. In at least one example, the extraction solvent may include a phosphate ester having a normal boiling point of at least about 180° C. In other embodiments, the extraction solvent may include a phosphate ester having a normal boiling point from about 185° C. to about 250° C., such as from about 190° C. to about 245° C.

In other embodiments, stripping is conducted at sub atmospheric pressure. In particular, the distillation column is operated at sub atmospheric pressure such that the absolute pressure within the column varies from about 25 mm Hg to about 90 mm Hg near the bottom of the column and from about 10 mm Hg to about 50 mm Hg near the top of the column. The temperature within the column may vary from about 180° C. to about 250° C. near the bottom of the column and from about 90° C. to about 105° C. near the top of the column.

According to another embodiment, the process further includes a step (c) of recycling at least a portion of the regenerable extraction solvent comprising the extraction solvent and contaminants to step (a). In some embodiments, the regenerable extraction solvent is treated prior to recycling in order to remove at least a portion of the contaminants. Such treatments may include, but are not limited to, vacuum and steam distillation, back extraction, absorption (for e.g., using a solid absorbent), and filtration. In one embodiment, the regenerable extraction solvent is treated by contacting the regenerable extraction solvent with an aqueous liquid extractant in an agitated extraction zone to produce a mixture comprising an aqueous extractant phase and an organic raffinate phase, the aqueous extractant phase being substantially immiscible with the regenerable extraction solvent.

As a result of contacting, contaminants which are water-soluble in the regenerable extraction solvent are thereby transferred to the aqueous liquid extractant and a two-phase mixture comprising an aqueous extract phase and an organic raffinate phase is produced. The organic raffinate phase contains the regenerable extraction solvent having a reduced concentration of contaminants. After separating the aqueous extractant and organic raffinate phases, the organic raffinate phase can be recycled back to step (a) for further contacting of additional gaseous mixtures for the absorption of maleic anhydride.

According to another embodiment, there is provided a system for the preparation and recovery of maleic anhydride. The system generally includes a reaction zone for preparing a gaseous mixture comprising maleic anhydride, an absorption zone for contacting the gaseous mixture and the extraction solvent of the present disclosure to transfer the maleic anhydride from the gaseous mixture to the extraction solvent, and a stripping zone to separate the maleic anhydride from the extraction solvent and optionally a regeneration zone.

The reaction zone is operable to catalytically oxidate a hydrocarbon and produce a gaseous mixture comprising maleic anhydride and then transfer the gaseous mixture to the absorption zone. The reaction zone generally includes a reactor containing a vanadium-phosphorus-oxygen catalyst and having an inlet for introducing a feed gas containing molecular oxygen and the hydrocarbon into the reactor and an outlet for discharging the gaseous mixture from the reactor. The reaction zone may further include one or more heat exchangers which are operable to cool the discharged gaseous mixture. In other embodiments, the reaction zone is substantially free of such heat exchangers.

The system also includes an absorption zone operable to contact the gaseous mixture with an extraction solvent of the present disclosure to transfer maleic anhydride from the gaseous mixture to the extraction solvent and produce an exhaust gas substantially free of maleic anhydride and a solvent effluent stream comprising the extraction solvent, maleic anhydride and contaminants. The absorption zone is further operable to transfer the solvent effluent stream to the stripping zone.

The absorption zone includes an absorber having a lower inlet for receiving the gaseous mixture, an upper inlet for receiving the extraction solvent, a vent line for exhausting the exhaust gas and an outlet for discharging the solvent effluent stream. The absorption zone also includes a solvent storage tank comprising the extraction solvent of the present disclosure. The solvent storage tank is operable to deliver the extraction solvent to the absorber. The tank includes one or more inlets to receive additional extraction solvent and the regenerable extraction solvent, and an outlet for withdrawing the extraction solvent.

The system also includes a stripping zone which is operable to separate the maleic anhydride from the solvent effluent stream to produce a crude maleic anhydride product and a regenerable extraction solvent comprising the extraction solvent and contaminants. The stripping zone is further operable to transfer the regenerable extraction solvent back to the absorption zone.

The stripping zone includes a distillation column having an inlet for receiving the solvent effluent stream, an upper outlet for discharging the crude maleic anhydride product and a lower outlet for discharging the regenerable extraction solvent.

The system optionally includes a regeneration zone operable to treat at least a portion of the regenerable extraction solvent to reduce the concentration of contaminants (i.e., contaminants which are water-soluble) therein. The regeneration zone is further operable to transfer the regenerable extraction having a reduced concentration of water-soluble contaminants to the absorption zone.

The regeneration zone includes an extractor having inlets for receiving the regenerable extraction solvent and an aqueous liquid extractant, an agitator for contacting and mixing the regenerable extraction solvent and aqueous liquid extractant to produce an aqueous extract phase containing water-soluble contaminants and an organic raffinate phase containing the regenerable extraction solvent and outlets for discharging the aqueous extract phase and organic raffinate phase, where the organic raffinate phase is recycled back to the absorption zone.

In still another embodiment, there is a provided a system for the recovery of maleic anhydride (i.e., the reaction zone is eliminated from the system described above) and generally includes an absorption zone for contacting a gaseous mixture comprising maleic anhydride and the extraction solvent to transfer the maleic anhydride from the gaseous mixture to the extraction solvent, a stripping zone to separate the maleic anhydride from the extraction solvent and optionally a regeneration zone to reduce the concentration of contaminants in the extraction solvent as described above.

A schematic diagram of the process and system 100 of the present disclosure is illustrated in the FIGURE. A feed gas mixture 101 comprising molecular oxygen and a hydrocarbon is introduced into reactor 103 containing an oxidation catalyst to partially oxidate the hydrocarbon to form a gaseous mixture comprising maleic anhydride. The feed gas mixture 101 can be produced by mixing a gas containing molecular oxygen, preferably air, and a gaseous hydrocarbon feedstock containing a hydrocarbon having not less than four carbons in a straight chain (for e.g., n-butane, 1-butene and 2-butene). Hydrocarbons contained in the feed gas mixture 101 are converted to maleic anhydride by contacting the feed gas mixture with a vanadium-phosphorus-oxygen catalyst at elevated temperatures in the reactor 103. The gaseous mixture 102 exits reactor 103 and may be subsequently cooled in heat exchanger 105. As discussed above, the extraction solvent of the present disclosure may allow for the subsequent absorption step to occur at a higher temperature than is achievable with the current state-of-the-art solvents and therefore the gaseous mixture 102 may not need to be cooled prior to being subjected to the absorption step. Therefore, in some embodiments heat exchanger 105 may be removed from system 100. Because such heat exchangers generally require significant operating expenses to maintain proper operation, removing the requirement for heat exchanger 105 can save significant operational expenses and related capital expenses.

The gaseous mixture 102 flows to an absorber 104, the absorber 104 containing means for contacting the gaseous mixture 102 with the extraction solvent 106. The absorber 104 may include a packing material (for e.g., saddles, rings etc.) for promoting gas/liquid contact and mass transfer of maleic anhydride from the gas phase to the liquid phase. Alternatively, the absorber 104 may include a tray column in which gas/liquid contact is affected on the trays. The gaseous mixture 102 may be introduced near the bottom of absorber 104, while the extraction solvent 106 from solvent storage tank 122 may be introduced near the top of the absorber 104. The gaseous mixture 102 and extraction solvent 106 flow counter-currently through absorber 104. Maleic anhydride and a portion of the oxidation by-products are absorbed in the extraction solvent 106 while the remaining oxidation by-products and inert gases in the gaseous mixture 102 are discharged through a vent at the top of absorber 104 as an exhaust gas 108 substantially free of maleic anhydride.

In some embodiments, the absorber 104 can operate at a temperature ranging from about 25° C. to about 260° C. In one embodiment, the amount of oxygen in the gaseous mixture 102 and the physical properties of the extraction solvent 106 are used to determine the maximum operating temperature of the absorber 104. In an alternative example, the maximum operating temperature of the absorber 104 is set to be equal to the flash point of the extraction solvent 106, including a reasonable safety margin of at least 10° C. Additionally, the minimum operating temperature of the absorber 104 is set at the melting point temperature of the extraction solvent 106. In at least some embodiments, the absorber 104 is operated at a pressure greater than atmospheric pressure. For instance, the pressure of the absorber 104 at a pressure up to about 50 psi above atmospheric pressure. When the absorber 104 is operated under pressure, the extraction solvent 106 described herein can absorb maleic anhydride at temperatures from about −60° C. to about 250° C.

In other embodiments, the ratio of extraction solvent 106 to gaseous mixture 102 introduced into the absorber 104 also varies within wide limits, but it may be preferred for reasons of economy that the ratio is set such that the solvent effluent stream 110 leaving the absorber 104 contains between about 5 wt. % and about 45 wt. % maleic anhydride, more preferably between about 15 wt. % and about 20 wt. %, based on the total weight of the solvent effluent stream. For example, for a gaseous mixture containing about 0.7 mole % maleic anhydride and about 7 mole % water, a ratio of extraction solvent 106 to gaseous mixture 102 is between about 0.07-0.3 kilograms extraction solvent per cubic meter of gaseous mixture.

The solvent effluent stream 110 can be removed near the bottom of absorber 104 and passed to distillation column 112 where the solvent effluent stream 110 is stripped of maleic anhydride in a stripping step to recover a crude maleic anhydride product 114 and a regenerable extraction solvent 116 comprising the extraction solvent and contaminants. The distillation column 112 includes a suitably sized housing having several vapor spaces and packed sections containing suitable packing materials. The solvent effluent stream 110 can be introduced into the bottom half of distillation column 112.

In some embodiments, the distillation column 112 may be operated at sub atmospheric pressure such that the absolute pressure within the distillation column 112 may vary from about 25-90 mm Hg near the bottom of the column 112 and from about 10-50 mm Hg near the top of the column 112. The temperature within the distillation column 112 may vary from about 180°-250° C. near the bottom of the column 112 to about 90°-105° C. near the top of column 112.

The crude maleic anhydride product 114 can be extracted as either a gas or a liquid near the top of distillation column 112. In embodiments where the crude maleic anhydride product 114 is extracted as a gas, it may be subsequently condensed in a condenser (not shown) to yield a liquid crude maleic anhydride product.

The regenerable extraction solvent 116 can be recovered from the bottom of the distillation column 112 and recycled through the system 100. In at least one embodiment, the regenerable extraction solvent 116 recovered from the bottom of the distillation column 112 can be recycled to solvent storage tank 122. In some embodiments, at least a portion of the contaminants present in the regenerable extraction solvent 116 can be removed prior to recycling by a treatment step in a regenerator.

Accordingly, in one embodiment at least a portion of the regenerable extraction solvent 116 is fed to an extractor 118 where the flow of regenerable extraction solvent is contacted with an aqueous liquid extractant under agitation. The aqueous liquid extractant is substantially immiscible with the regenerable extraction solvent and in some embodiments the aqueous liquid extractant comprises deionized water. As a result of contacting (and agitating) the regenerable extraction solvent with the aqueous extractant, water-soluble contaminants contained in the regenerable extraction solvent are transferred to the aqueous liquid extractant and a two-phase mixture comprising an aqueous extract phase and an organic raffinate phase is produced, the organic raffinate phase containing the regenerable extraction solvent having a reduced concentration of contaminants.

The extractor 118 may comprise a suitably-sized pressure vessel having an inlet for the regenerable extraction solvent, an inlet for the aqueous liquid extractant and an outlet for withdrawing the organic raffinate phase from the extractor. Extractor 118 may comprise a propeller for agitating the mixture of the aqueous and organic phases within the extraction zone to promote intimate turbulent contact between the phases and mass transfer of water-soluble contaminants from the regenerable extraction solvent to the aqueous liquid extractant.

The extraction may occur at moderate process conditions (including, without limitation, temperatures of from about 40° C. to about 80° C., and pressures of from about 20 psig to about 100 psig). The ratio of the volumetric proportions of aqueous liquid extractant to regenerable extraction solvent charged to the extractor 118 may be from about 1:1 to about 1:10, or from about 1:4 to about 1:6.

The separated aqueous extract phase containing the water-soluble contaminants that have been transferred from the regenerable extraction solvent can be discharged from the system 100 as waste while the separated organic raffinate phase can be discharged from the extractor 118 and combined with any regenerable extraction solvent 116 that was bypassed around extractor 118 or directly transferred (not shown) to solvent storage tank 122. In an alternative embodiment (not shown), the regenerable extraction solvent (treated or untreated) can directly re-enter the absorber column 104.

In one embodiment, some extraction solvent may decompose during the absorption and stripping steps. In such cases, fresh extraction solvent may be added to solvent storage tank 122 through a solvent make-up line 124. Phosphate esters having a low water solubility are generally preferred to prevent large losses of maleic anhydride product and of the extraction solvent as it circulates through system 100. As such, the solubility of the improved extraction solvent in water is preferably less than about 100 mg/L. In at least one example, solubility of the improved extraction solvent in water is less than about 50 mg/L. In at least one additional example, solubility of the improved extraction solvent in water is less than about 25 mg/L or less than about 10 mg/L.

Low boiling by-products and a portion of contaminants contained in the solvent effluent stream transferred to distillation column 112 are vented from the top of the distillation column 112 as gas stream 120. In at least some instances, a small amount of maleic anhydride may also be present in gas stream 120. In such instances, the maleic anhydride can be recovered from gas stream 120 via extraction in a scrubber (not shown), and the scrubbed stream containing maleic anhydride can be returned to system 100 or combined with the crude maleic anhydride product 114 exiting the system 100.

While the foregoing is directed to embodiments of the present disclosure, other and further embodiments of the disclosure may be devised without departing from the basic scope thereof, and the scope thereof is determined by the claims that follow.

1. A process for recovering maleic anhydride from a gaseous mixture containing the maleic anhydride, the process comprising a step (a) of contacting the gaseous mixture with an extraction solvent comprising a phosphate ester wherein the contacting comprises transferring at least a portion of the maleic anhydride from the gaseous mixture to the extraction solvent.

2. The process of claim 1, wherein the phosphate ester is a compound having the formula 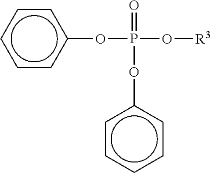
where R³ is a linear or branched alkyl group having up to 22 carbon atoms, an aryl group, or an -aryl-(C₁-C₁₀)alkyl group.

3. The process of claim 2, wherein the phosphate ester is a mono alkyl diphenyl phosphate ester where R³ is a linear or branched alkyl group containing up to 22 carbon atoms.

4. The process of claim 3, wherein the mono alkyl phosphate ester comprises 2-ethylhexyl diphenyl phosphate, nonyl diphenyl phosphate, iso-octyl diphenyl phosphate, 2-methylpentyl diphenyl phosphate, 2-n-propylheptyl diphenyl phosphate, 2-butyloctyl diphenyl phosphate, 2-ethylbutyl diphenyl phosphate, isodecyl diphenyl phosphate or a mixture thereof.

5. The process of claim 2, wherein R³ is an -aryl-(C₁-C₄)alkyl group.

6. The process of claim 5, wherein R³ is tolyl, cresyl, xylyl, t-butylphenyl or isopropylphenyl.

7. The process of claim 1, wherein the extraction solvent further comprises an organic solvent.

8. The process of claim 1, wherein the extraction solvent is substantially free of an organic solvent.

9. The process of claim 1, wherein the contacting step (a) is conducted at a temperature within a range from about −60° C. to about 250° C.

10. The process of claim 1, further comprising a step (b) of subjecting the solvent effluent stream obtained in step (a) to stripping to produce a first stream comprising a crude maleic anhydride product and a regenerable extraction solvent substantially free of maleic anhydride and comprising the extraction solvent and contaminants.

11. The process of claim 1, wherein step (b) is conducted in a distillation column operating at a temperature of between about 180° C. to about 250° C.

12. The process of claim 11, wherein step (b) is conducted at sub atmospheric pressure.

13. The process of claim 1, further comprising a step (c) of recycling at least a portion of the regenerable extraction solvent comprising the extraction solvent and contaminants to step (a).

14. The process of claim 13, wherein the regenerable extraction solvent comprising the extraction solvent and contaminants is treated prior to recycling to reduce the concentration of contaminants in the regenerable extraction solvent.

15. The process of claim 14, wherein the regenerable extraction solvent is treated by contacting the regenerable extraction solvent with an aqueous liquid extractant to transfer at least a portion of the contaminants from the regenerable extraction solvent to the aqueous liquid extractant.

16. The process of claim 15, wherein the contacting produces a two-phase mixture comprising an aqueous extract phase comprising the at least portion of contaminants and an organic raffinate phase comprising the regenerable extraction solvent having the reduced concentration of contaminants.

17. A system for the preparation and recovery of maleic anhydride, the system comprising a reaction zone operable to prepare a gaseous mixture comprising maleic anhydride, an absorption zone operable to contact the gaseous mixture and an extraction solvent comprising a phosphate ester to transfer the maleic anhydride from the gaseous mixture to the extraction solvent, and a stripping zone operable to separate the maleic anhydride from the extraction solvent wherein the absorption zone comprises a solvent storage tank comprising the extraction solvent.

18. A system for the recovery of maleic anhydride, the system comprising an absorption zone operable to contact a gaseous mixture comprising maleic anhydride and an extraction solvent comprising a phosphate ester to transfer the maleic anhydride from the gaseous mixture to the extraction solvent, and a stripping zone operable to separate the maleic anhydride from the extraction solvent, wherein the absorption zone comprises a solvent storage tank comprising the extraction solvent.
